(19) United States
(12) Patent Application Publication
(10) Pub. No.: US 2015/0133397 A1
(43) Pub. Date: May 14, 2015

(54) METHOD OF REDUCING FASTING INSULIN LEVELS IN MTHFR GENE DEFICIENT SUBJECTS WITH NORMAL TO SLIGHTLY ELEVATED HEMOGLOBIN A1C

(71) Applicants: Jeffrey Lynn Barringer, Lowell, AR (US); Stephen Albert Smith, Alexandria, LA (US); Jerry Golden, Fairhope, AL (US)

(72) Inventors: Jeffrey Lynn Barringer, Lowell, AR (US); Stephen Albert Smith, Alexandria, LA (US); Jerry Golden, Fairhope, AL (US)

(21) Appl. No.: 14/455,256

(22) Filed: Aug. 8, 2014

Related U.S. Application Data
(60) Provisional application No. 61/866,415, filed on Aug. 15, 2013.

(51) Int. Cl. A61K 31/519; A61K 31/714

(52) U.S. Cl. CPC A61K 31/519; A61K 31/714
USPC 514/52; 514/249

(57) ABSTRACT

A method of treatment for reducing elevated fasting insulin levels of subjects with a MTHFR gene deficiency is provided. The method includes first determining whether a subject has an MTHFR gene deficiency. Once a subject has been found to have the MTHFR gene deficiency, the fasting insulin levels of the subject are determined. If the subject has a high fasting insulin level, the subject is administered an active folate.

### CROSS-REFERENCE TO RELATED APPLICATION

This application claims the benefit of priority of U.S. provisional application No. 61/866,415, filed Aug. 15, 2013, the contents of which are herein incorporated by reference.

### BACKGROUND OF THE INVENTION

The present invention relates to insulin treatment and, more particularly, to a method of reducing fasting insulin levels in MTHFR gene deficient subjects.

Methylenetetrahydrofolate reductase (MTHFR) is the rate-limiting enzyme in the methyl cycle, and it is encoded by the MTHFR gene. Methylenetetrahydrofolate reductase catalyzes the conversion of 5,10-methylenetretrahydrofolate to 5-methyltetrahydrofolate, a cosubstrate for homocysteine remethylation to methionine. Genetic variation in this gene may influence susceptibility to occlusive vascular disease, neural tube deficiencys, dementia, colon cancer, and acute leukemia, because mutations in this gene are associated with methylenetetrahydrofolate reductase deficiency. Further, subjects with a MTHFR gene deficiency have a propensity to incur damage associated with fasting elevated insulin levels.

Currently subjects with a MTHFR gene deficiency are prone to elevated fasting insulin levels because the MTHFR gene deficiency can cause functional deficiency of folate and B12. A functional deficiency of folate and B12 can lead to elevated fasting insulin levels and endothelial dysfunction. Subjects with a normal HbA1C (hemoglobin A1C) to slightly elevated A1C can develop elevated fasting insulin levels as a result of the MTHFR gene deficiency. The MTHFR can also increase fasting insulin levels in both the prediabetic and the diabetic. Elevated fasting insulin levels are defined as equal to or greater than 10 μU/ml according to Berkley Heart Labs and Health Diagnostics Laboratories Incorporated.

The HbA1C test is a common blood test used to diagnose type 1 and type 2 diabetes and then to gauge how well the subject is managing diabetes or the subjects risk of becoming diabetic. The A1C test result reflects the subjects average blood sugar level for the past two to three months. Specifically, the A1C test measures what percentage of the subject's hemoglobin is coated with sugar (glycated). The higher the subject's A1C level, the poorer the blood sugar control and the higher the risk of complications associated with elevated fasting insulin levels. According to the American Diabetes Association guidelines a HbA1C of between 4 and 5.6% falls within normal parameters, HbA1C between 5.7 and 6.4% are considered slightly elevated and indicate a prediabetic state as where HbA1C of greater than 6.5% is considered diabetic.

Subjects that have the MTHFR gene deficiency may not show a positive result for the A1C test, and therefore are not treated for diabetes. However, as mentioned above, MTHFR deficient subjects may have high levels of fasting insulin even if the test for the A1C is negative. Therefore, MTHFR deficient subjects have a propensity to acquire elevated fasting insulin levels if they are left untreated.

As can be seen, there is a need for a method of treatment of MTHFR deficient subjects that have elevated fasting levels of insulin.

### SUMMARY OF THE INVENTION

In one aspect of the present invention, a method of treatment for reducing fasting insulin levels of subjects with a MTHFR gene deficiency comprises: determining whether a subject has an MTHFR gene deficiency; determining fasting insulin levels for a subject that has the MTHFR gene deficiency; and administering an active folate to a subject with the MTHFR gene deficiency and a high fasting insulin level.

These and other features, aspects and advantages of the present invention will become better understood with reference to the following drawings, description and claims.

### BRIEF DESCRIPTION OF THE DRAWINGS

FIG. 1 is a flow chart of an embodiment of the present invention; and

FIG. 2 is a diagram of active folate being processed within the body.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes the use of active folate to lower fasting insulin levels of humans/animals with MTHFR gene deficiency. Administering active folate to MTHFR gene deficient subjects improves endothelial function by increasing Nitric Oxide production resulting in improved fasting insulin levels. Active folate is able to bypasses the commonly occurring MTHFR gene deficiency that lead to elevated fasting insulin levels. The active folate may be in a biologically stable form to be administered to a subject with a MTHFR gene deficiency. Methyl B12 and/or vitamin D3 could be added to improve function and absorption resulting in improved response.

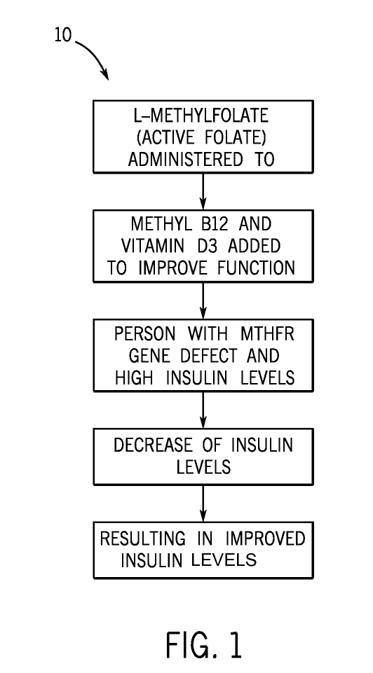

Referring now to FIG. 1, the present invention includes a method of treatment for reducing fasting insulin levels of subjects with a MTHFR gene deficiency. The method includes first determining whether a subject has an MTHFR gene deficiency. Once a subject has been found to have the MTHFR gene deficiency, the fasting insulin levels of the subject are determined. If the subject has a high fasting insulin level, the subject is administered an active folate.

The insulin levels may be tested to determine whether the subject is at risk for elevated fasting insulin levels. If the fasting insulin level is 10 μU/ml or greater, the chances that the subject will acquire damage from high fasting insulin levels are greater. Therefore, for subjects that have fasting insulin levels of 10 μU/ml or greater may be provided the active folate, which in turn reduces the subjects fasting insulin levels so that the subject is at a lower risk for damage from high fasting insulin.

The active folate may be administered at doses of 0.01 mcg up to 30 mg once or twice daily depending on elevation of the fasting insulin level. The administration of the active folate to a subject with an MTHFR gene deficiency may increase or restore the subject's production of Nitric Oxide. The increased production of Nitric Oxide improves the endothelial function which results in the improved fasting insulin levels.

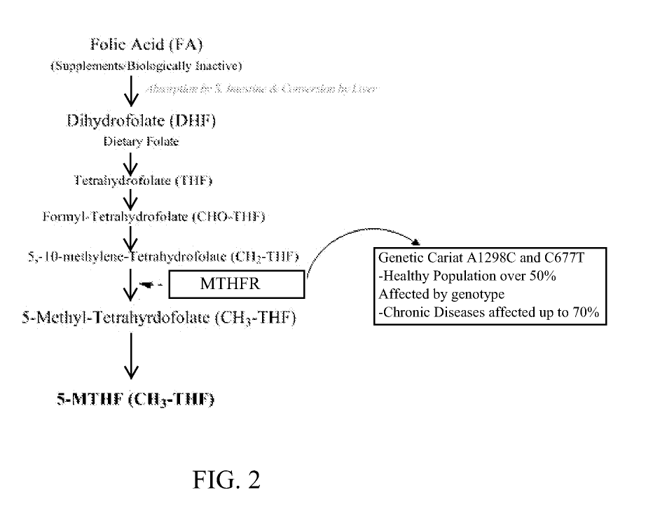

Biologically active folate requires no further breakdown into a functional form and therefore bypasses the MTHFR gene deficiency. Folic acid is synthetic and requires a complex 5 step conversions to its functional form and therefore is affected by MTHFR gene deficiency. Further, dietary folate requires a complex 4 step breakdown and is also affected by the MTHFR gene deficiency. FIG. 2 is a diagram of the active folate being processed within the body.

The present invention includes the administration of active folate, such as L-methylfolate or any of its derivatives administered alone or in combination with other ingredients, synthetic or natural the L-methyfolate comprises a natural isomer of reduced folate comprising at least one of (6S)-tetrahydrofolic acid, 5-methyl-(6S)-tetrahydrofilic acid, 5-formyl-(6S)-tetrahydrofolic acid, 10-formyl-(6R)-tetrahydrofolic acid, 5,10-methenyl-(6R)-tetrahydrofolic acid, 5-formyl-(6S)-tetrahydrofolic acid, 5-formimino-(6S)-tetrahydrofolic acid, levomefolic, 5 MTHF, 5 methyltetrahydrofolate and their poly-glutamyl derivatives.

It should be understood, of course, that the foregoing relates to exemplary embodiments of the invention and that modifications may be made without departing from the spirit and scope of the invention as set forth in the following claims.

### EXAMPLE 1

The test subject was a 24 year old female. The test subject was tested for an MTHFR gene deficiency and tested positive for MTHFR677 tt. The test subject was given an A1C test and had an HbA1C of 4.9% blood sugar level. The test subject was given a fasting insulin test. The test subject had a fasting insulin level of 40 μU/ml. The test subject was administered 3 mgs of L-methylfolate with 2 mgs methyl B12 once daily. The subject was retested 6 months later and HbA1C was 4.9% and the fasting insulin level was 9 μU/ml.

### EXAMPLE 2

The test subject was a 28 year old male. The test subject was tested for an MTHFR gene deficiency and tested positive for MTHFR677 tt. The test subject was given an A1C test and had an HbA1C of 5.3% blood sugar level. The test subject was given a fasting insulin test. The test subject had a fasting insulin level of 100 μU/ml. The test subject was administered 15 mgs of L-methylfolate with 2 mgs methyl B12 once daily. The subject was retested 6 months later and HbA1C was 5.3% and the fasting insulin level was 11 μU/ml.

What is claimed is:

1. A method of treatment for reducing elevated fasting insulin levels of subjects with a MTHFR gene deficiency comprising:
determining whether a subject has an MTHFR gene deficiency;
determining fasting insulin levels for a subject that has the MTHFR gene deficiency; and
administering an active folate to a subject with the MTHFR gene deficiency and a high fasting insulin level.

2. The method of claim 1, wherein a high fasting insulin level is a fasting insulin level of 10 μU/ml or greater.

3. The method of claim 1, wherein the administration of the active folate to the subject with the MTHFR gene deficiency and the high fasting insulin level increases production of Nitric Oxide.

4. The method of claim 1, wherein the administration of the active folate to the subject with the MTHFR gene deficiency and the high fasting insulin level lowers the fasting insulin level within the subject.

5. The method of claim 1, wherein the active folate is L-methylfolate.

6. The method of claim 5, wherein the L-methyfolate comprises a natural isomer of reduced folate comprising at least one of (6S)-tetrahydrofolic acid, 5-methyl-(6S)-tetrahydrofilic acid, 5-formyl-(6S)-tetrahydrofolic acid, 10-formyl-(6R)-tetrahydrofolic acid, 5,10-methenyl-(6R)-tetrahydrofolic acid, 5-formyl-(6S)-tetrahydrofolic acid, 5-formimino-(6S)-tetrahydrofolic acid, levomefolic, 5 MTHF, 5 methyltetrahydrofolate and the poly-glutamyl derivatives.

7. The method of claim 1, wherein the active folate is combined with at least one of methyl B12 and vitamin D3.

8. The method of claim 1, wherein the active folate is administered at doses from 0.01 mcg to 30 mg.
